(19) United States
(12) Patent Application Publication
(10) Pub. No.: US 2011/0144000 A1
(43) Pub. Date: Jun. 16, 2011

(54) ANTIMICROBIAL POLYPEPTIDE

(76) Inventors: Arne Egesten, Hjarup (SE); Mattias Collin, Lund (SE); Helena M. Linge, Port Washington, NY (US)

(21) Appl. No.: 12/745,479

(22) PCT Filed: Nov. 28, 2008
(86) PCT No.: PCT/SE08/00667

§ 371 Date: May 28, 2010

Related U.S. Application Data
(60) Provisional application No. 60/991,987, filed on Dec. 3, 2007.

(30) Foreign Application Priority Data
Nov. 30, 2007 (SE) 0702668-5

(51) Int. Cl. A61K 38/16; C07K 7/06; C07K 7/08; C07K 14/00; A61K 38/08; A61K 38/10; A61P 31/04

(52) U.S. Cl. 514/2.4; 530/330; 530/329; 530/328; 530/327; 530/326; 530/325; 530/324; 514/21.8; 514/21.7; 514/21.6; 514/21.5; 514/21.4; 514/21.3; 514/21.2

(57) ABSTRACT

A molecule has a first isolated peptide as shown in SEQ ID NO: 1 or part thereof or a peptide having at least 78% homology to SEQ ID NO:1 conjugated to a second peptide. The second peptide is an amphipatic peptide with an alpha-helical structure or a linear cationic peptide and the first and second peptides have a length of from about 5 to 100 amino acid residues. The molecule is used in medicine as well as for the manufacturing of a medicament for the treatment of a mammal in need thereof, such as for the treatment of a bacterial disease or disorder.

### FIELD OF THE INVENTION

The invention relates to a molecule comprising a first isolated peptide as shown in SEQ ID NO:1 or part thereof or a peptide having at least 78% homology to SEQ ID NO:1 conjugated to a second peptide wherein said second peptide is an amphipatic peptide with an alphahelical structure or a linear cationic peptide and wherein said first and second peptide have a length of from about 5 to 100 amino acid residues. The invention also relates to the use of said molecule in medicine as well as for the manufacturing of a medicament for the treatment of a mammal in need thereof; such as for the treatment of a bacterial disease or disorder.

### BACKGROUND OF THE INVENTION

Peptides having different origin and structure have been found to be active against a number of organisms such as different kinds of bacteria.

The chemokines make up a family of peptides containing conserved cysteine motifs at their NH₂-terminus, i.e. XC, CC, CXC, and CX₃C, respectively, where “X” is a non-conserved amino acid residue (Baggiolini, 2001). These molecules are responsible for leukocyte trafficking and activation, in both health and disease. In addition to chemotactic activity, some chemokines possess antibacterial activity but not all of them do. This is probably due to the different amino acid sequences as well as different structures (Cole et al., 2001 and Yang et al., 2003). Granulocyte chemotactic protein (GCP)-2/CXCL6 belongs to the human ELR-positive CXC chemokine family and the corresponding gene is localized on chromosome 4 together with other members of the CXC chemokine family (Proost et al., 1993; 1999; Rovai et al., 1997). The NH₂-terminal glutamic acid-leucine-arginine (ELR) motif is characteristic of CXC-chemokines interacting with the receptors CXCR1 and CXCR2. These receptors are expressed on a variety of cells, for example neutrophils, monocytes/macrophages, T and NK cells, mast cells, and endothelial cells (Baggiolini, 2001). In the case of leukocytes, GCP-2/CXCL6 binding to the receptors causes cellular activation, chemotaxis, and sometimes, depending on context, execution of effector functions (Baggiolini, 2001). In the case of endothelial cells, GCP-2/CXCL6 causes a mitogenic response, resulting in angiogenesis (Gijsbers et al., 2005).

The amino acid sequence of CXCL6 is similar to the amino acid sequence of CXCL5 and both have a pI value below 10. According to Yang et al., 2003, no activity of CXCL6 motif was found against bacteria and this was thought to be due to CXCL6 having a low pI. In table 1 in the paper by Yang et al., 2003, it appears that a pI value of at least around 10 is necessary in order to obtain anti-bacterial activity. Accordingly, Cole et al., 2001 disclosed that CXCL5, the protein showing the closest sequence homologies to CXCL6, has no activity against bacteria. Thus, the literature at the time of the invention indicated that neither CXCL5 nor CXCL6 have activity against bacteria.

The CXCL6 chemokine has earlier been described as useful in the prevention or repair of cartilage defects (WO 2005/014026). Said document does not suggest that the chemokine has effect against bacteria.

### SUMMARY OF THE INVENTION

Despite the fact that the literature at the time showed that neither CXCL5 nor CXCL6 have activity against bacteria, the inventors chose to investigate CXCL6 again. They found in their experiments that the CXCL6 shows activity, which was unexpected.

Thus, the invention relates to the finding that small peptides with unique amino acid sequences each on their own show an antibacterial effect against a number of bacteria. However, when conjugated to each other these peptides show an increased activity. Thereby it is possible to produce new molecules wherein the combination gives rise to an improved peptide with an even higher activity.

In a first aspect the invention relates to a molecule comprising a first isolated peptide as shown in SEQ ID NO:1 or part thereof or a peptide having at least 78 homology to SEQ ID NO:1 conjugated to a second peptide b) or c), wherein b is an amphipathic peptide with an alpha-helical structure and c is a linear cationic peptide and wherein said first and second peptide have a length of from about 5 to 100 amino acid residues. By combining a specific set of isolated peptides with each other a new improved peptide is obtained having new improved properties, such as having an increased antimicrobial effect.

In a second aspect the invention relates to a molecule as defined above for use in medicine as well as use of the molecule for the manufacturing of a medicament for the treatment of a disease or disorder.

In a third aspect the invention relates to a method of treating a mammal having a bacterial disease or disorder, comprising administering to a patient a therapeutically effective amount of a pharmaceutical composition as mentioned above.

### BRIEF DESCRIPTION OF THE DRAWINGS

FIG. 1 a and b. GCP-2/CXCL6 (SEQ ID NO:3) as well as part of CXCL6 (SEQ ID NO:1 and 2) are antibacterial towards both Gram-positive and G-negative bacterial pathogens. Viable count assays were performed using Streptococcus pyogenes, Streptococcus dysgalactiae, sub species equisimilis, Staphylococcus aureus, Escherichia coli, and Pseudomonas aeruginosa, cultured to mid-log phase and then incubated with GCP-2/CXCL6 in concentrations ranging from 0.01-1 μM.

FIG. 2. Predicted structure of GCP-2/CXCL6 (SEQ ID NO:3) and antibacterial activity in the NH₂- and COOH-terminal regions respectively.

FIG. 3 shows the effect of the two related ELR-positive CXC chemokines CXCL5 (SEQ ID NO:4) and CXCL7 (SEQ ID NO:5).

FIG. 4 shows the amino acid sequence alignment between CXCL, 5, 6 and 7.

### DETAILED DESCRIPTION OF THE INVENTION

### Definitions

In the context of the present application and invention the following definitions apply:

The term “nucleotide sequence” is intended to mean a consecutive stretch of three or more regions of nucleotide sequences. The nucleotides can be of genomic. DNA, cDNA, RNA, semisynthetic or synthetic or a mixture thereof. The term includes single and double stranded forms of DNA or RNA.

The term “analogue thereof” is intended to mean that part of or the entire polypeptide of SEQ ID NO:2 is based on non-protein amino acid residues, such as aminoisobutyric acid (Aib), norvaline gamma-aminobutyric acid (Abu) or ornitihine. Examples of other non protein amino acid residues can be found at http://www.hort.purdue.edu/rhodcv/hort640c/polyam/po00008.htm.

The term “homology” is understood as the degree of identity between two sequences indicating a derivation of the first sequence from the second. The homology may suitably be determined by means of computer programs known in the art such as GAP provided in the GCG program package (Program Manual for the Wisconsin Package, Version 8, August 1994, Genetics Computer Group, 575 Science Drive, Madison, Wis., USA 53711) (Needleman, S. B. and Wunsch, C. D., (1970), Journal of Molecular Biology, 48, 443-453. The following settings for amino acid sequence comparison are used: GAP creation penalty of 3.0 and GAP extension penalty of 0.1. The relevant part of the amino acid sequence for the homology determination is the mature polypeptide.

The term “amphipathic” is intended to mean the distribution of hydrophilic and hydrophobic amino acid residues along opposing faces of an α-helix structure, β-strand, linear, circular, or other secondary conformation, as well as along the peptide primary structure, which result in one or several domains of the molecule being predominantly charged and hydrophilic and the other being predominantly hydrophobic.

The term “substitution” is intended to mean that a specific amino acid residue has been replaced with another amino acid residue, such as a natural or synthetic amino acid residue.

In the present context, amino acid names and atom names are used as defined by the Protein DataBank (PNB) (www.pdb.org), which is based on the IUPAC nomenclature (IUPAC Nomenclature and Symbolism for Amino Acids and Peptides (residue names, atom names etc.), Eur J Biochem., 138, 9-37 (1984) together with their corrections in Eur J Biochem., 152, 1 (1985). The term “amino acid” is intended to indicate an amino acid from the group consisting of alanine (Ala or A), cysteine (Cys or C), aspartic acid (Asp or D), glutamic acid (Glu or E), phenyl-alanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Tip or W) and tyrosine (Tyr or Y), or derivatives thereof.

### Molecule of the Invention

The invention relates to improved molecules, wherein said molecules have increased activity against bacteria when two peptides are conjugated together.

In a first embodiment the invention relates to a molecule comprising a first isolated peptide as shown in SEQ ID NO:1 or part thereof or a peptide having at least 78% homology to SEQ ID NO:1 conjugated to a second peptide, wherein said second peptide is an amphipatic peptide with an alpha-helical structure or is a linear cationic peptide and wherein said first and second peptide together have a length of from about 5 to 100 amino acid residues. Accordingly, said first peptide may show 80, 85, 90, 91, 92, 93, 94, 95, 96, 97, 98 or 99% homology to SEQ ID NO:1. One example being that said second peptide is as shown in SEQ ID NO:2 or a peptide having at least 80% homology to SEQ ID NO:2 and another example being that the first peptide is as shown in SEQ ID NO:1 and said second peptide is as shown in SEQ ID NO:2 or SEQ ID NO:3.

The molecule of the invention may have a length of for example 5, 6, 7, 8, 9, 10, 11, 12, 13, 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 amino acid residues, as long as it retains its anti-bacterial activity. Examples of specific length are 10-100. 10-50, 10-25, 15-25, 15-20 or 20-25 amino acid residues.

The peptides of the invention may be in a substantially isolated form. It will be understood that the polypeptide may be mixed with carriers or diluents which will not interfere with the intended purpose of the polypeptide and still be regarded as substantially isolated.

The amino acid residues of the invented molecule may be modified either in the first or in the second peptide or in both of the peptides to include non-naturally occurring amino acids or to increase the stability of the compound. When the peptides are produced by synthetic means, such amino acids may be introduced during production. The peptides may also be modified following either synthetic or recombinant production. The amino acid residues of the invented peptides may be modified by at least one substitution such as 2, 3, 4 or 5 substitutions in said first or said second peptide or both of the peptides.

Peptides for use in the invention may also be produced using D-amino acids. In such cases the amino acids will be conjugated in reverse sequence in the C to N orientation. This is conventional in the art for producing such polypeptides.

A number of side chain modifications are known in the art and may be made to the side chains of the polypeptides, subject to the polypeptides retaining any further required activity or characteristic as may be specified herein.

It will also be understood that the peptides used in the invention may be chemically modified, e.g. post-translationally modified. For example, they may be glycosylated, phosphorylated or comprise modified amino acid residues. They may be modified by the addition of a signal sequence to promote insertion into the cell membrane.

The peptides of the invention may also be derivatised, such as by HIS-tag or modified to assist with their isolation or purification. Thus, in one embodiment of the invention, the peptide of is derivatised or modified by addition of a ligand which is capable of binding directly and specifically to a separation means. Alternatively, the peptide is derivatised or modified by addition of one member of a binding pair and the separation means comprises a reagent that is derivatised or modified by addition of the other member of a binding pair. Any suitable binding pair can be used. In a preferred embodiment the peptide is derivatised or modified by addition of one member of a binding pair, such as histidine-tagged or biotin-tagged. Typically the amino acid coding sequence of the histidine or biotin tag is included at the gene level and the proteins are expressed recombinantly in E. coli. The histidine or biotin tag is typically present at one end of the polypeptide, either at the N-terminus or at the C-terminus. The histidine tag typically consists of six histidine residues, although it can be longer (typically up to 7, 8, 9, 10 or 20 amino acids) or shorter (for example 5, 4, 3, 2 or 1 amino acids). Furthermore, the histidine tag may contain one or more amino acid substitutions, such as conservative substitutions as defined above.

The addition of a histidine tag means that the peptide will bind with a high affinity to the separation means used in the invention when said means comprises a reagent containing chelating groups on its surface which carry a nickel, copper or zinc ion. The histidine tag binds strongly to these metal ions.

The addition of a biotin tag means that the peptide will bind with high affinity to the separation means used in the invention when said means comprises a reagent comprising streptavidin. The biotin tag binds strongly to streptavidin.

Any suitable separation means may be used, such as a solid support. Examples of solid supports include cross-conjugated agarose beads, or similar, which may be used as the matrix in an affinity chromatography column. Alternatively the solid support may comprise a suitable silica-based material or polystyrene. In one embodiment the solid support may comprise a plastic container such as a microtiter plate or equivalent, to which the peptide can be directly adsorbed.

Alternative separation means include reagents comprising antibodies specific to the peptide, which may be generated by methods standard in the art. Antibodies in this sense include a monoclonal antibody, polyclonal antibodies, a single chain antibody, a chimeric antibody, or a humanized antibody. The antibody may be an intact immunoglobulin molecule or a fragment thereof such as a Fab, F(ab′)2 or Fv fragment. If more than one antibody is present, the antibodies preferably have different non-overlapping determinants such that they may bind to the peptide simultaneously. The antibody may be bound to a solid support or may be labelled or conjugated to another chemical group or molecule to assist with their separation or isolation. For example, typical chemical groups include fluorescent labels such as Fluorescein (FITC) or Phycoerythrin (PE), or tags such as biotin.

The peptides for use in the present invention may be isolated from any suitable organism that expresses suitable peptides, one example being the CXCL6 peptide (SEQ ID NO.3) or a variant of an CXCL6 peptide. Alternatively the invented peptides may be produced synthetically. The peptides for use in the invention may also be prepared as fragments of such isolated peptides. Further, the peptides may also be made synthetically or by recombinant means. For example, a recombinant peptide may be produced by transfecting mammalian cells in culture with an expression vector comprising a nucleotide sequence encoding the polypeptide operably conjugated to suitable control sequences, culturing the cells, extracting and purifying the peptide produced by the cells. For example, full-length or fragments of for example CXCL6 may be expressed in Escherichia coli using the GST Gene Fusion System (Amersham-Pharmacia Biotech, Uppsala, Sweden). Oligonucleotide primers (with introduced restriction enzyme sites) corresponding to the preferred fragment of the human CXCL6 gene can be amplified using polymerase chain reaction (PCR) from cDNA generated from mRNA prepared from human cells. These fragments are digested with the appropriate restriction enzymes, ligated into pGEX-5×−3 generating plasmid pGEXcxcl6, that is used to transform E. coli BL21(DE3)pLys. pGEXndoS/BL21(DE3)pLys is induced with 0.1 mM isopropyl β-D-thiogalactopyranoside. After induction, bacteria are lysed using BugBuster™ (Novagen) and the GST-CXCL6 fusion proteins are purified on Glutathione-Sepharose®. The GST tag is removed using factor Xa according to protocols (Amersham-Pharmacia Biotech), and residual factor Xa is removed using Xarrest™-agarose (Novagen). This results in a preparation of recombinant CXCL6 or fragments thereof (rCXCL6) that is homogenous as assessed by SDS-PAGE and Western blot using CXCL6-specific antibodies. Prior to in vivo experiments protein samples are sterile-filtered through a 0.2 μm filter (Millipore). Purified proteins are stored at −80° C. in phosphate buffered saline.

Alternatively, PCR products encoding full-length or fragment of CXCL6 may be cloned into the pcDNA3.1 expression vector using the pcDNA 3.1 Directional TOPO expression kit (Invitrogen, Carlsbad, Calif.) to obtain the expression plasmids. The expression plasmids are then transiently transfected into HeLa cells using the Geneporter transfection system (Gene Therapy Systems, San Diego, Calif.) according to manufacturer's protocol. ProBond™ nickel-chelating resin (Invitrogen) is used to purify His6-tagged recombinant hRasGRP4 from the transfectants. Approximately 1×107 transfectants are placed in 4 ml of 20 mM sodium phosphate, pH 7.4, buffer containing 500 mM sodium chloride and multiple protease inhibitors (Roche Diagnostics). Each cell suspension is lysed by two freeze-thaw cycles using liquid nitrogen and a 42° C. water bath. Liberated nuclear DNA is sheared by passing the resulting preparation through an 18-gauge needle four times. Cellular debris is removed by a 5-min centrifugation step at 4° C. and at ˜44,000×g. The resulting recombinant protein-enriched supernatant is incubated with nickel-charged agarose resin for 1 h at 4° C. to ensure efficient binding of the His6-tagged recombinant protein. After the equilibration step, the resin is centrifuged in a “Spin” column at 800×g for 2 min. The non-bound material is discarded, and the column is washed extensively with 500 mM sodium chloride and 20 mM sodium phosphate, pH 6.0, to remove weakly associated protein. Five ml of 50 mM imidazole elution buffer is applied, and the resulting recombinant protein-enriched eluate is concentrated to ˜0.5 ml with a Centriplus-50 (Millipore, Bedford, Mass.) filtering device having a 50-kDa cut-off membrane.

Examples of microorganisms that the invented pharmaceutical composition may be used against includes, both Gram positive and Gram-negative bacteria such as Enterococcus faecalis, Eschericia coli, Pseudomonas aeruginosa, Proteus mirabilis, Streptococcus pneumoniae, Streptococcus pyogenes, Staphylococcus aureus, Finegoldia magna and Helicobacter pylori. Other microorganisms of interest include, but are not limited to Citrobacter sp., Klebsiella sp., Enterobacter sp., Morganella, Providencia, Listeria sp., Salmonella sp., Serratia sp., Shigella sp., Yersinia sp., Pasteurella sp., Vibrio sp., Campylobacter sp., Haemophilus sp., Bordetella sp., Brucella sp., Neisseria sp., Legionella sp., Mycoplasma sp., mycobacterium sp., Propiomibacterium sp. and Chlamydia sp.

The invented molecule may be used in medicine, such as in a pharmaceutical composition or alone. The molecule may also be used for the manufacturing of a medicament for the treatment of a disease or disorder, such as a disease or disorder caused by one or more bacteria.

Such a pharmaceutical composition comprises the above defined molecule and a pharmaceutically acceptable carrier, excipient, diluent or buffer. Such a pharmaceutical composition as well as the molecule may be used in medicine. “Pharmaceutically acceptable” means a non-toxic material that does not decrease the effectiveness of the biological activity of the active ingredients, i.e., the antimicrobial peptide(s). Such pharmaceutically acceptable buffers, carriers or excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000).

The term “buffer” is intended to mean an aqueous solution containing an acid-base mixture with the purpose of stabilising pH.

The term “diluent” is intended to mean an aqueous or non-aqueous solution with the purpose of diluting the peptide in the pharmaceutical preparation. The diluent may be one or more of saline, water, polyethylene glycol, propylene glycol, ethanol or oils (such as safflower oil, corn oil, peanut oil, cottonseed oil or sesame oil).

The excipient may be one or more of carbohydrates, polymers, lipids and minerals. Examples of carbohydrates include lactose, sucrose, mannitol, and cyclodextrines, which are added to the composition, e.g., for facilitating lyophilisation. Examples of polymers are starch, cellulose ethers, cellulose carboxymethylcellulose, hydroxypropylmethyl cellulose, hydroxyethyl cellulose, ethylhydroxyethyl cellulose, alginates, carageenans, hyaluronic acid and derivatives thereof, polyacrylic acid, polysulphonate, polyethylenglycol/polyethylene oxide, polyethyleneoxide/polypropylene oxide copolymers, polyvinylalcohol/polyvinylacetate of different degree of hydrolysis, and polyvinylpyrrolidone, all of different molecular weight, which are added to the composition, e.g., for viscosity control, for achieving bioadhesion, or for protecting the lipid from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

The pharmaceutical compositions according to the invention may be administered locally or systemically. Routes of administration include topical, ocular, nasal, pulmonar, buccal, parenteral (intravenous, subcutaneous, and intramuscular), oral, vaginal and rectal. Also administration from implants is possible. Suitable preparation forms are, for example, granules, powders, tablets, coated tablets, (micro) capsules, suppositories, syrups, emulsions, microemulsions, defined as optically isotropic thermodynamically stable systems consisting of water, oil and surfactant, liquid crystalline phases, defined as systems characterised by long-range order but short-range disorder (examples include lamellar, hexagonal and cubic phases, either water- or oil continuous), or their dispersed counterparts; gels, ointments, dispersions, suspensions, creams, aerosols, droplets or injectable solution in ampule form and also preparations with protracted release of active compounds, in whose preparation excipients, diluents or carriers are customarily used as described above. The pharmaceutical composition may also be provided in bandages, plasters or in sutures or the like.

The pharmaceutical compositions will be administered to a patient in a pharmaceutically effective dose. By “pharmaceutically effective dose” is meant a dose that is sufficient to produce the desired effects in relation to the condition for which it is administered. The exact dose is dependent on the, activity of the compound, manner of administration, nature and severity of the disorder, age and body weight of the patient Different doses may be needed. The administration of the dose can be carried out both by single administration in the form of an individual dose unit or else several smaller dose units and also by multiple administrations of subdivided doses at specific intervals

The pharmaceutical compositions of the invention may be administered alone or in combination with other therapeutic agents, such as antibiotic, anti-inflammatory or antiseptic agents such as anti-bacterial agents. Examples are penicillins, cephalosporins, carbacephems, cephamycins and carbapenems. Other examples are quinolones, hydrogen peroxide or fucidic acid.

The invented molecule or pharmaceutical composition may be used in a method of treating a mammal having a bacterial disease or disorder, comprising administering to a patient a therapeutically effective amount of the molecule or pharmaceutical composition.

The present invention concerns both humans and other mammal such as horses, dogs, cats, cows, pigs, camels, among others. Thus the methods are applicable to both human therapy and veterinary applications.

Examples of diseases are topical applications such as diabetic ulcers, chronic infested wounds and impetigo. Other examples are inhalation/spray compositions useful for pharyngitis, nasal carriers as well as immuno-compromised patients such as cystic fibrosis, chronic obstructive pulmonary disease (COPD), transplant patients, HIV/AIDS and rheumatoid arthritis patients.

Further examples are sutures, indwelling catheters, surgical wound edges, prosthetic joints such as hips, gastro-intestinal inflammation or infection as well as shoe cuts and burns.

Following examples are intended to illustrate, but not to limit, the invention in any manner, shape, or form, either explicitly or implicitly.

### EXAMPLES

### Example 1

### Chemicals and Reagents

Recombinant human chemokine GCP-2/CXCL6 and affinity-purified polyclonal rabbit antibodies against GCP-2/CXCL6 directed thereto, were from PeproTech, London, United Kingdom.

### Bacterial Strains and Growth Conditions

The GAS strain AP1 (40/58) of M1 serotype was from the World Health Organization Collaborating Centre for Reference and Research on Streptococci, Prague, Czech Republic. Streptococcus pyogenes (strain AP1) and Streptococcus Dysgalactiae, sub species equisimilis (strain G41), Staphylococcus aureus (strain 5120), Pseudomonas aeruginosa (strain 1553 initially isolated from a chronic venous ulcer) and Escherichia coli (strain 37.4) were routinely grown in Todd-Hewitt (TH) liquid medium in 5% CO₂ and at 37° C. The bacterial strains were obtained from Division of Infection Medicine, Department of Clinical Sciences, Lund University, BMC B14, Tornavagen 10, SE-221 84 Lund, Sweden.

### Example 2

### Molecular Modelling

A predictive structure of GCP-2/CXCL6 was modelled using the VMD 1.8.5. application (University of Illinois). For helical wheel depiction, EMBOSS Pepwheel (www.tcdb.org/progs/pepwheel.php) was used. The synthetic peptides GPV-50 (SEQ ID NO:2; GPVSAVLTELRCTCLRVTLRVNPKTIGKLQVFPAGPQCSKVEVVASLKNG) and APF-19 (SEQ ID NO:1; APFLKKVIQKILDSGNKKN) derived from the sequence of the human CXCL6 holopeptide were of 95% purity (Genscript Corporation, Piscataway, N.J., USA).

### Structural Prediction and Physiochemical Properties of GCP-2/CXCL6

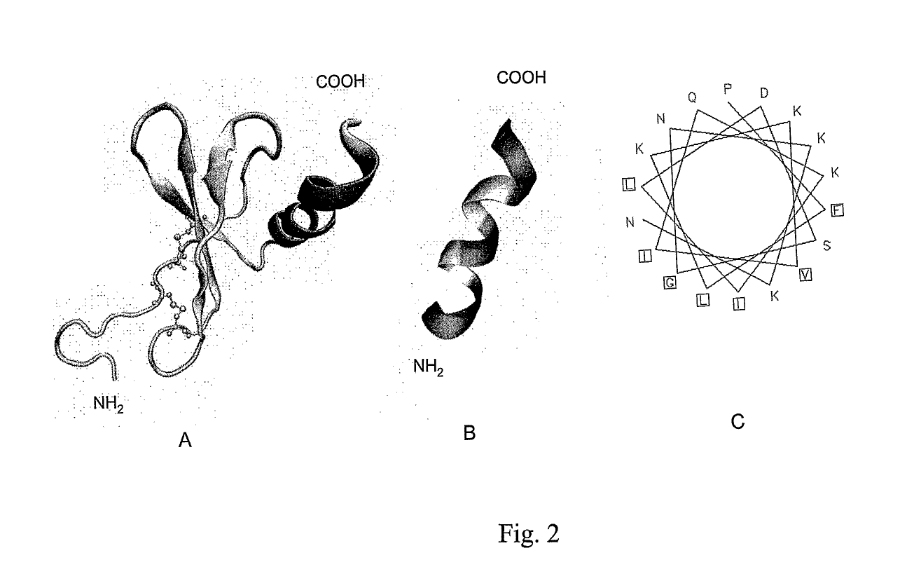

In order to locate the antibacterial region of GCP-2/CXCL6 (SEQ ID NO:3), a predicative model based on known structures of other members of the chemokine family was made (FIG. 2). GCP-2/CXCL6 contains the typical CXC motif which forms disulphide bonds with the cysteines bonds with the cysteines nr 12-38 and 14-54 respectively. The N terminus is an unstructured region, followed by three anti-parallel β-sheets and a short α-helix When displaying the aa:s by class, the C-terminal helix gives an amphipathic appearance. This was confirmed depicting a helical wheel which gives a view of a helix from a protein sequence looking down the axis of the helix. This was confirmed by entering aa residues 56-77 to Pepwheel (FIG. 2).

### Example 3

### Bactericidal Assay

### Viable Count Assay

Bacteria were cultivated to mid-log phase (OD₆₂₀ 0.4) in Todd-Hewitt broth, washed, and diluted in incubation buffer (10 mM Tris-Hcl pH 7.4 containing 5 mM glucose). Pseudomonas aeruginosa was grown for 18 hours at 37° C. 50 μl of bacteria (1×10⁶ colony forming units (cfu)/mL) were incubated together with various concentrations of peptide or buffer alone for 1 h at 37° C. In order to quantify bactericidal activity, serial dilutions of the incubation mixtures were plated on TH agar. The number of resulting cfu:s were related to bacteria incubated with buffer alone and the results expressed as % killing.

### GCP-2/CXCL6 Possesses Antibacterial Activity

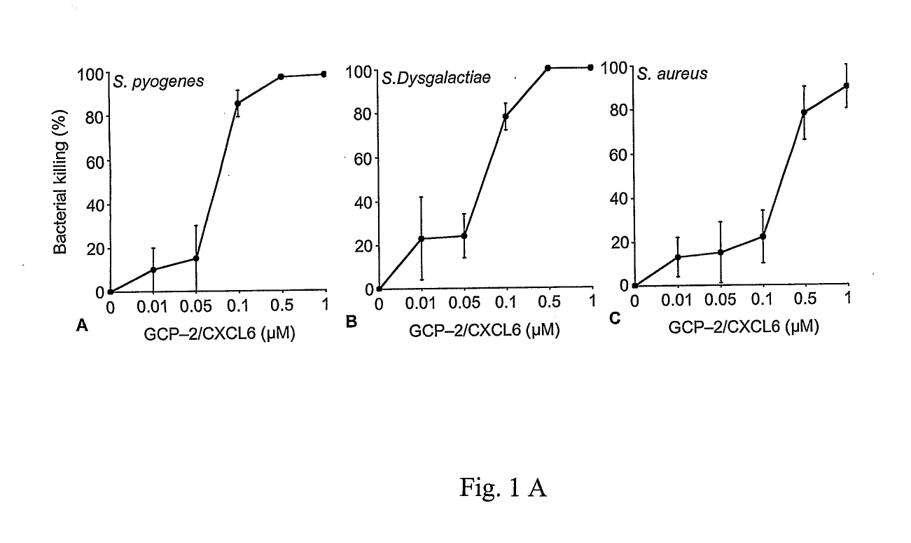

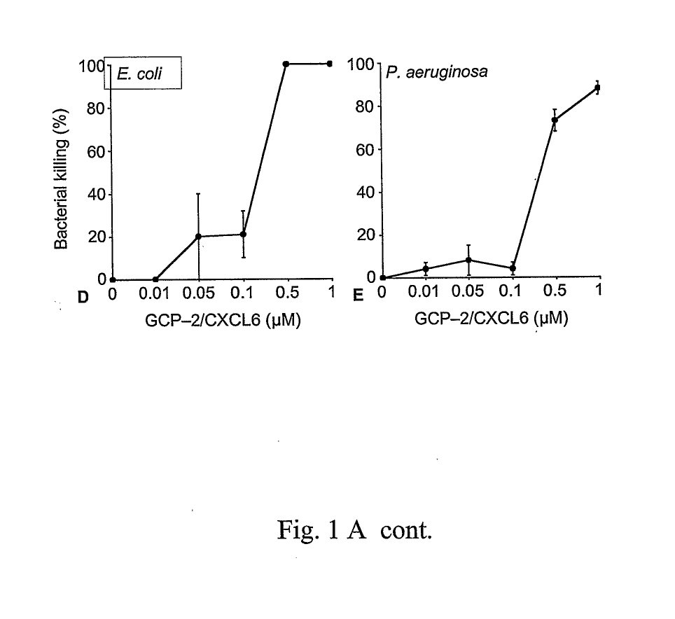

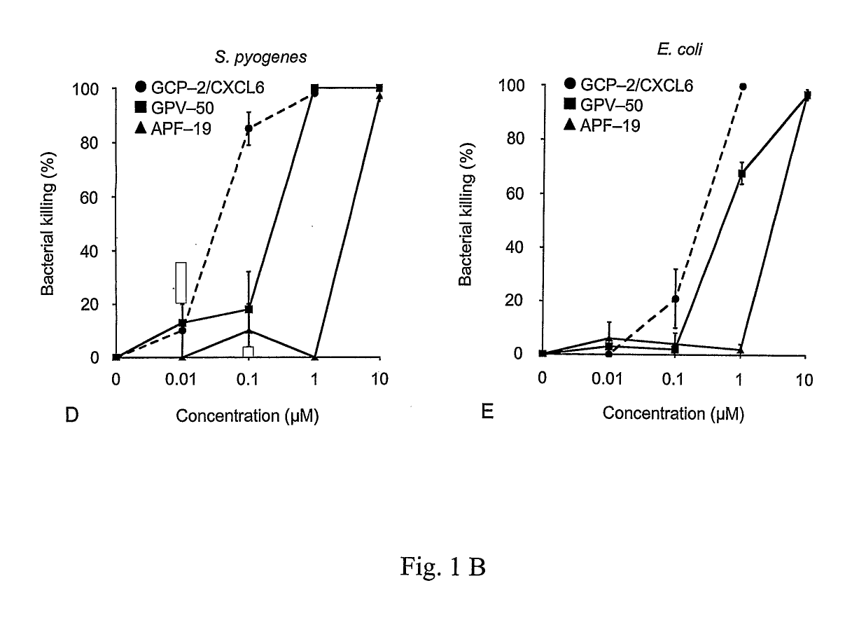

GCP-2/CXCL6 (SEQ ID NO:3) as well as parts of CXCL6, i.r., SEQ ID NO:1 and 2 being part of CXCL6 was examined for antibacterial activity against a panel of bacteria relevant during infections of dermis and mucosal surfaces. The bacteria included Gram-positive (Group A streptococci, group G streptococci, and S. aureus) and G-negative (E. coli and P. aeruginosa) bacteria (FIG. 1 a). GCP-2/CXCL6 had a bactericidal effect on all investigated bacterial species at below micromolar concentrations. To delineate what regions of the GCP-2/CXCL6 molecule that are responsible for antibacterial activity, a 50 amino acid fragment of the NH₂-terminal region (GPV-50) of GCP-2/CXCL6 and a 19 amino acid fragment (APF-19) of the COOH-terminal region (a region with a putative amphiphatic alpha-helical structure), was compared with the GCP-2/CXCL6 holopeptide. Antibacterial activity was investigated against S. pyogenes and E. coli. GPV-50 showed higher antibacterial activity than APF-19, both being less antibacterial than the holopeptide (FIG. 1b).

### Example 4

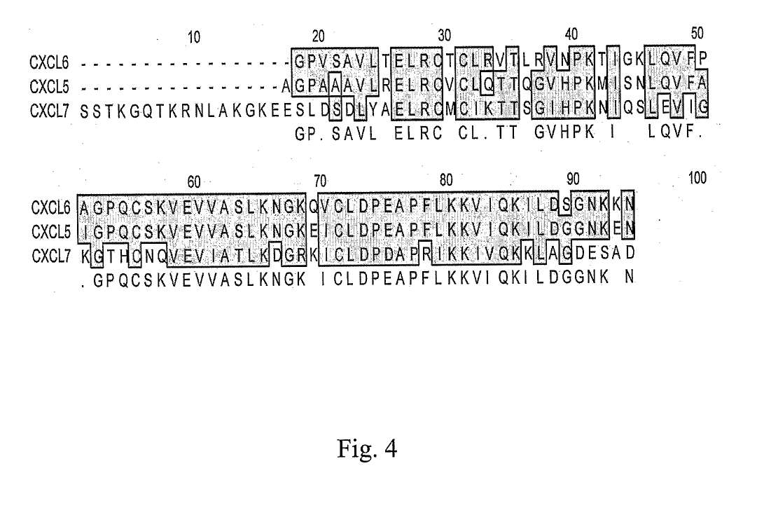

The amino acid sequences of CXCL6 (SEQ ID NO:3), 5 (SEQ ID NO:4) and 7 (SEQ ID NO:5) were compared to each other (see FIG. 4). Clustal W sequence alignment of the mature forms of CXCL 6, 5 and 7 show that. CXCL5 is 75% identical and 81% similar to CXCL6 and CXCL7 is 34% identical and 55% similar to CXCL6.

### Example 5

### Comparison of the Antibacterial Activity of ENA-78/CXCL5 and GCP-2/CXCL6

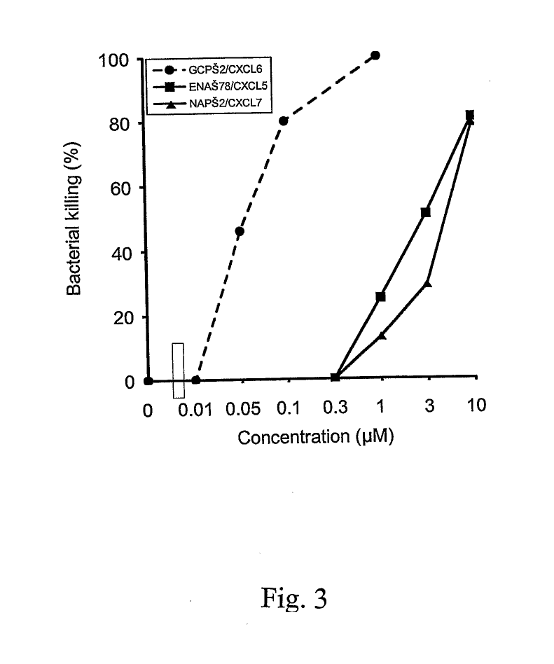

The antibacterial activity of the ELR-positive CXC-chemokines ENA-78/CXCL5 and GCP-2/CXCL6 chemokine was compared using the viable count assay described in Example 3 in experiments with S. pyogenes (AP1 strain). The results are shown in FIG. 3.

### Example 6

### Antibacterial Activity Against E. faecalis

The anti-bacterial activity of the GCP-2/CXCL6 (SEQ ID NO:3) against the gram-positive, facultatively anaerobic bacteria Enterococcus faecalis was determined using the previously described experimental set-up (See Example 3).

| | |
|---|---|
| MBC50 | 0.46 ± 0.03 μM (mean, ±SEM) |
| MBC90 | 0.90 ± 0.00 μM |

### Example 7

### Antibacterial Activity Against N. gonorrhoeae

The anti-bacterial activity of the GCP-2/CXCL6 (SEQ ID NO:3) against the gram-negative bacteria Neisserria gonorrhoea was determined using the previously described experimental set-up (See Example 3).

| | |
|---|---|
| MBC50 | 0.3 ± 0.1 μM (mean, ±SEM) |
| MBC90 | 0.8 ± 0.1 μM |

### REFERENCES

Baggiolini M. Chemokines in pathology and medicine. J Intern Med. 2001; 250:91-104.
Cole A M, Ganz T, Liese A M, Burdick M D, Liu L, Strieter R M. Cutting edge: IFN-inducible ELR-CXC chemokines display defensin-like antimicrobial activity. J. Immunol. 2001; 167: 623-627.
Gijsbers K, Gouwy M, Struyf S, Wuyts A, Proost P, Opdenakker G, Penninckx F, Ectors N, Geboes K, Van Damme J. GCP-2/CXCL6 synergizes with other endothelial cell-derived chemokines in neutrophil mobilization and is associated with angiogenesis in gastrointestinal tumors. Exp Cell Res. 2005; 303:331-342.
Proost P, De Wolf-Peeters C, Conings R, Opdenakker G, Billiau A, Van Damme J. Identification of a novel granulocyte chemotactic protein (GCP-2) from human tumor cells. In vitro and in vivo comparison with natural forms of GRO, IP-10, and IL-8. J Immunol. 1993; 150:1000-1010.
Rovai L E, Herschman H R, Smith J B. Cloning and characterization of the human granulocyte chemotactic protein-2 gene. J Immunol. 1997; 158:5257-5266.
Yang D, Chen Q, Hoover DM, Staley P, Tucker K D, Lubkowski J, Oppenheim J J. Many chemokines including CCL20/MIP-3alpha display antimicrobial activity. J. Leukoc. Biol. 2003; 74: 448-455.

1. A molecule comprising
a) a first isolated peptide as shown in SEQ ID NO: 1 or part thereof or a
i. peptide having at least 78% homology to SEQ ID NO: 1 conjugated to a
ii. second peptide b) or c), wherein
b) is an amphipatic peptide with an alphahelical structure and
c) is a linear cationic peptide and wherein said first and second peptide have a length of from about 5 to 100 amino acid residues.

2. The molecule according to claim 1, wherein said isolated peptide shows 80% homology to SEQ ID NO: 1.

3. The composition according to claim 2, wherein said isolated peptide shows 90% homology to SEQ ID NO: 1.

4. The molecule according to claim 3, wherein said isolated peptide shows 99% homology to SEQ ID NO: 1.

5. The molecule according to claim 4, wherein said second peptide is as shown in SEQ ID NO:2 or a peptide having at least 80% homology to SEQ ID NO:2.

6. The molecule according to claim 4, wherein said second peptide is as shown in SEQ ID NO:3 or a peptide having at least 80% homology to SEQ ID NO:3.

7. The molecule according to claim 1, wherein the first peptide is as shown in SEQ ID NO: 1 and said second peptide is as shown in SEQ ID NO:2.

8. The molecule according to claim 1, wherein said first and/or said second peptide is modified by at least one substitution.

9. A method for using the molecule according to claim 1, comprising using in medicine.

10. A method for using molecule according to claim 1, comprising using in veterinary medicine.

11. A pharmaceutical composition comprising the molecule according to claim 1 and a pharmaceutically acceptable carrier, excipient, diluent or buffer.

12. A pharmaceutical composition comprising the molecule according to claim 1, further comprising at least one other therapeutic agent.

13. A method of using the molecule according to claim 1, comprising manufacturing of a medicament for the treatment of a disease or disorder.

14. A method of using the molecule according to claim 1 for the manufacturing of a medicament for the treatment of a disease or disorder caused by one or more bacteria.

15. The use of the molecule according to claim 1, comprising manufacturing of a medicament for the prevention of a disease or disorder.

16. A method of using the molecule according to claim 1, comprising manufacturing of a medicament for the prevention of a disease or disorder caused by bacteria.

17. A method of treating a mammal having a bacterial disease or disorder, comprising administering to a patient a therapeutically effective amount of a pharmaceutical composition according to claim 9.


SEQUENCE LISTING (5 sequences)

SEQ ID NO: 1 - protein, 19 aa, artificial
  CXCL6 peptide
APFLKKVIQKILDSGNKKN

SEQ ID NO: 2 - protein, 50 aa, artificial
  CXCL6 peptide
GPVSAVLTELRCTCLRVTLRVNPKTIGKLQVFPAGPQCSKVEVVASLKNG

SEQ ID NO: 3 - protein, 77 aa, artificial
  GCP-2/CXCL6 peptide
GPVSAVLTELRCTCLRVTLRVNPKTIGKLQVFPAGPQCSKVEVVASLKNGKQVCLDPEAPFLKKVIQKILDSGNKKN

SEQ ID NO: 4 - protein, 78 aa, artificial
  CXCL5 peptide
AGPAAAVLRELRCVCLQTTQGVHPKMISNLQVFAIGPQCSKVEVVASLKNGKEICLDPEAPFLKKVIQKILDGGNKEN

SEQ ID NO: 5 - protein, 94 aa, artificial
  CXCL7 peptide
SSTKGQTKRNLAKGKEESLDSDLYAELRCMCIKTTSGIHPKNIQSLEVIGKGTHCNQVEVIATLKDGRKICLDPDAPRIKKIVQKKLAGDESAD